(19) United States
(12) Patent Application Publication
(10) Pub. No.: US 2008/0208150 A1
(43) Pub. Date: Aug. 28, 2008

(54) Container with Security Closure and Kit for Samples of Urine and the Like

(76) Inventor: Andre de Lima Castro, Rio de Janeiro (BR)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 12/065,618

(22) PCT Filed: Jul. 7, 2006
(86) PCT No.: PCT/BR06/00138

§ 371 Date: Mar. 4, 2008

(30) Foreign Application Priority Data
Sep. 12, 2005 (BR) P10503648-8

(51) Int. Cl. A61M 1/00

(52) U.S. Cl. 604/323

(57) ABSTRACT

A container having a security closure is described, of the type comprising a hollow body (9) with a mouth or neck (10) defining an opening to the interior of the body, the mouth having an outer screw thread and also a first series of ratchet elements (2). A closure (3) adaptable to the mouth of the container so as to close the opening in the body has a closed end and an outer skirt, the outer skirt (18) being formed with an inner screw thread that co-operates with the outer screw thread on the mouth. The closure is also formed with a second series of ratchet elements (21) so that, on screwing the closure onto the mouth, the co-operation between the ratchet elements (12, 21) of the first and second series permits closure of the container, but prevents unscrewing if the closure.

The present invention refers to a container with a security closure developed principally, but not exclusively, for samples of urine or the like for later analysis, so as to prevent any attempt at adulteration or exchange of the samples before their removal from the container in the laboratory. In particular, the container is suitable for doping tests applied to football players, athletes participating in official competitions and the like.

The invention also refers to an anti-doping kit including the container with the security closure.

### PRIOR ART

The art of security closures is not new. Prior to the present invention many documents have been published which disclose containers having screw closures as well as ratchet type mechanisms to prevent or complicate their opening. In particular, such a technique has been developed for the creation of “child-proof” containers for pharmaceutical products whereby, once the cap has been screwed onto a container containing a medicine, a ratchet mechanism prevents unscrewing unless the cap is axially lifted or pressed down to disengage the ratchet teeth and the cap is at the same time rotated to unscrew it from the container. Being a relatively complicated procedure, children are unable to open the container.

Other examples of containers using generally similar principles cannot be opened without rupturing a safety ring that incorporates the ratchet teeth of either the lid or the container.

Examples of such types of container can be found in U.S. Pat. Nos. 3,944,101; 3,963,139; 4,345,691; and 4,752,013.

Such prior art containers, however, do not enjoy a high degree of security, partly because the purpose is more to complicate opening or generally to show that the container has already been opened once rather than to guarantee, with all certainty, that the contents have not been tampered with between the moment when they were placed in the container and an authorised opening thereof. In this sense all prior art devices suffer from some type of serious drawback should one be seeking a higher degree of security against tampering. For example, the ratchet teeth on the container are normally positioned on the lower outer region of the neck of the container and cooperate with similar elements on the lower inner region of the closure. Access to the ratchet mechanism is thus relatively simple and it is not a difficult task to “fiddle” the ratchet elements so as to open the container without leaving any sign of violation. This facility effectively eliminates the possibility of using such containers for collecting and transporting samples of urine of athletes, for example, for doping tests.

Kits especially developed for use in collecting samples for such type of test are known. For example, U.S. Pat. No. 5,690,246 discloses a system that uses a first container for the sample, with its respective cap without any security feature, and a second outer container into which the first container is placed. The outer container has an opening for receiving the sample containing container and a hinged closure is then fitted into the opening using a ratchet type system that permits inward axial movement but prevents axial removal of the closure. Apart from the drawback of requiring an additional outer container for ensuring security, this system uses the ratchet engagement as the only closure mechanism and, especially taking into consideration that it is made of plastic, this represents a considerable risk of tampering by unauthorised third parties.

### OBJECTS OF THE PRESENT INVENTION

The principal object of this invention is to provide a container having a closure that incorporates a high security mixed screw and ratchet closure system.

Another object of the invention is to provide a container of the above type that is simple and inexpensive to manufacture.

A further object is to provide a security container provided with a simple and inexpensive means that will permit the removal of the contents of the container without damaging its lid and its security closure characteristic, thus allowing checking of tampering or not of the security system, even after removal of the contents.

An even further object of the invention is to provide a kit for doping tests for athletes, which will allow the simultaneous taking of two samples in different distinct containers, using containers with security closures in accordance with this invention.

### BRIEF DESCRIPTION OF THE PRESENT INVENTION

The first aspect of this invention relates to a container with a security closure of the type comprising a hollow body having a mouth or neck defining an opening to the interior of the body, the mouth having an outer thread and also a first series of ratchet elements. A closure adaptable to the mouth of the container for closing the opening in the body has a closed end and an external skirt, such external skirt being formed with an inner thread co-operable with the outer thread of the mouth. The closure is also formed with a second series of ratchet elements so that, when the closure is screwed onto the mouth, the co-operation between the ratchet elements of the first and second series permits closure of the container but prevents unscrewing of the closure.

According to the invention, the ratchet elements of the first series are distributed circumferentially around the inner surface of the mouth; the closure is provided with an inner skirt such that, when the container is closed, the mouth fits between the outer and inner skirts of the closure; and the ratchet elements of the second series are distributed circumferentially around the outer surface of the inner skirt.

Preferably and with a view to cause irreparable and visibly apparent damage in the event of an attempt to violate the container by unscrewing the closure, the mouth is connected to the body of the container by means of a weakened circumferential region that is less resistant to twisting forces between the mouth and the body than those necessary to unscrew the closure from the mouth, against the resistance provided by the ratchet elements.

In the presently preferred embodiment of the invention, each ratchet element of one of the series is flexible so as to allow passage of the ratchet elements of the other series when the closure is screwed onto the mouth of the container but, due to their abutment against the ratchet elements of the other series, resists bending when unscrewing is attempted. In this embodiment, the ratchet elements of the second series comprise teeth formed around the external surface of the inner skirt and the ratchet elements of the first series are flexible tongues, each tongue being integral at one end with the inner surface of the mouth and having a free end that inter-engages with a sloping side of successive teeth to flex the tongue and thus to allow screwing on of the closure, and a stop side of a respective tooth then prevents bending of the tongue so as not to allow unscrewing of the closure.

It is also preferred for the body to be formed, in a location remote from the mouth, with a weakened easily broken upstanding region to provide an outlet opening for the contents of the body without requiring removal of the closure. The weakened upstanding region is conveniently located in a recessed part of the base of the container.

According to a second aspect of the invention, a kit for collecting samples of urine comprises a urine receiving tray having a base with two interspaced orifices, each orifice leading to a lower tubular portion projecting downwardly from the lower surface of the base and each tubular portion having an internal thread.

The kit further includes two containers with closures, of the type described above, the outer thread on the mouth of each container cooperating with the inner thread on either one of the lower tubular portions of the tray, thus allowing the two containers to be mounted below the tray so as simultaneously to receive the sample.

### BRIEF DESCRIPTION OF THE DRAWINGS

The present invention will now be described in greater detail, by way of example, with reference to the accompanying drawings, in which:

FIG. 1 is a perspective view of the elements of a doping test kit according to a presently preferred embodiment of the present invention;

FIG. 2 is a vertical central section taken through one of the closures shown in FIG. 1;

FIG. 3 is a vertical central section taken through one of the containers shown in FIG. 1;

FIG. 4 is a perspective view of one of the closures, in an inverted position so as to show its interior; and

FIG. 5 is a detail showing the ratchet locking system of the closure onto the container, seen from above.

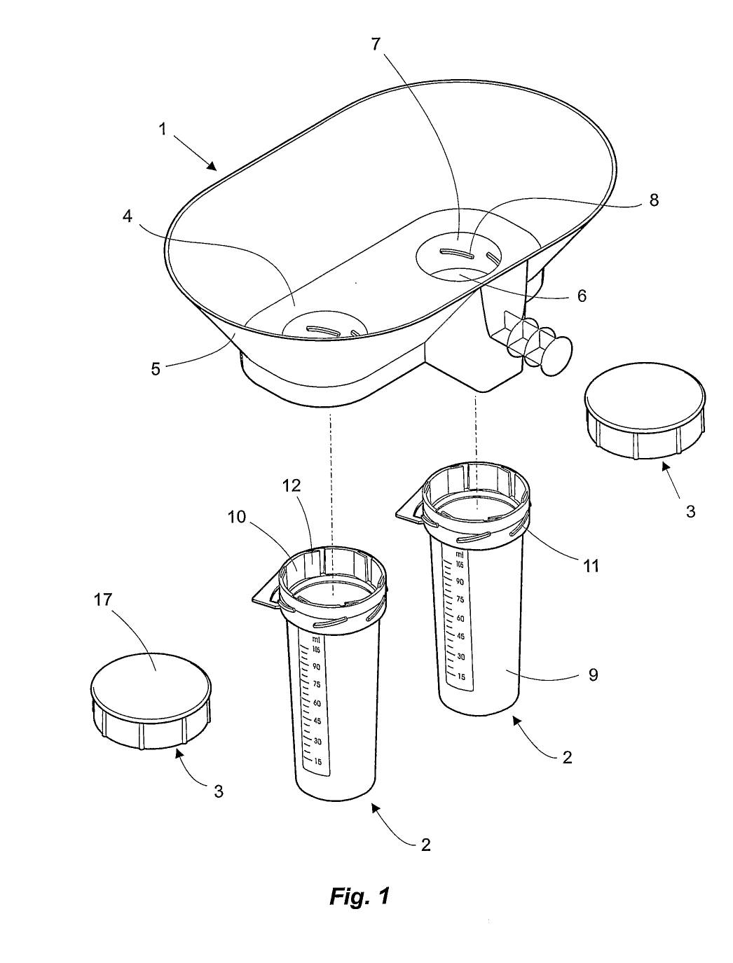

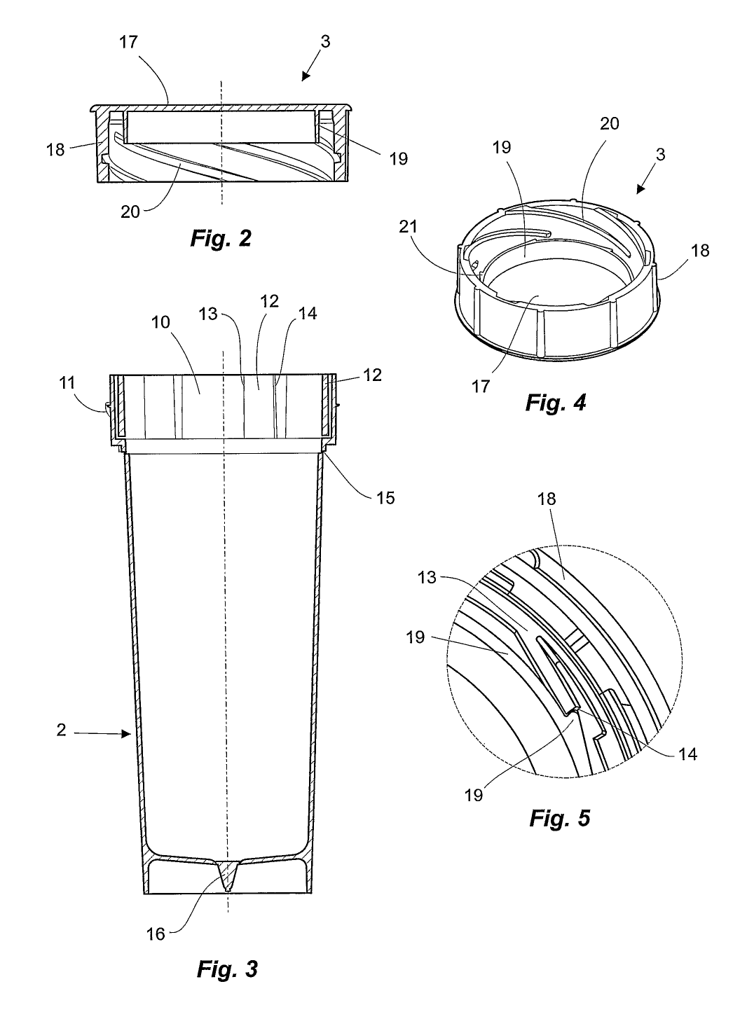

### DETAILED DESCRIPTION OF THE DRAWINGS

Referring now to the drawings which show the presently preferred embodiment of the present invention, a doping test kit is composed of a tray 1, two collector containers 2 and two security closure lids 3. Each of these five components is a one-piece plastic moulded part of a relatively simply shape, which ensures simple and economic manufacture.

Tray 1 has a bottom or base 4, a peripheral side wall 5 and two openings 6 formed in the base 4. Around each opening 6, on the underside of base 4, there is a cylindrical tubular part 7 formed with an inner screw thread 8 for coupling one of the containers 2 to the tray, as will be described later.

The tray also has a lateral grip to facilitate handling.

Each container 2 comprises a main body 9 the upper portion of which is formed with a mouth 10 having an outer screw thread 11 adapted to co-operate with the tubular part 7 that projects downwardly from tray 1.

Mouth 10, which defines the opening designed to receive a sample of urine from tray 1 through one of openings 6, is also provided, on its inner surface, with a series of ratchet elements in the form of tongues 12. Each tongue 12, which has a substantially flattened rectangular shape, has a base end 13 integral with the inner surface of mouth 10, and an opposite free end 14. The tongues are extended in the clockwise direction (when seen from above) and are generally peripherally arranged around the inner surface of the mouth, but at an angle somewhat less than 90° (about 80°) with respect to radii of the mouth that pass through their base ends 13. Depending on the material used for manufacturing the containers (for example, polyethylene, polycarbonate or acrylic), the width of each tongue 12, measured axially of mouth 10, may occupy a greater or lesser extension of the inner surface of the mouth. This will determine the resistive strength provided by the ratchet system.

It will also be seen that the junction 15 between body 9 and mouth 10 of each container 2 is weaken by having a reduced thickness, the purpose of which will be mentioned later.

Finally, with respect to each container 2, the base or bottom of body 9 is recessed and centrally formed with a weakened protuberance 16. The weakening of protuberance 16 is such that it is easily ruptured, either manually or by using a suitable tool, so as to open an outlet at the bottom of the container and thus permit removal of the urine sample therein when the doping test is to be carried out. Removal of the sample in this way is doubly advantageous. Firstly, it provides a very simple, practical and hygienic way of removing the sample, without there being any necessity to tip the container. Secondly, such “authorized” opening of the container in no way interferes with the closure of mouth 10 by means of closure lid 3 which facilitates later inspection should suspicion of tampering be raised later.

Each lid 3 has a top panel 17, an external cylindrical skirt 18 extending downwardly form the outer periphery of top panel 17, an inner cylindrical skirt 19, also extending downwardly from panel 17, but over a lesser extension, an inner screw thread 20 on external skirt 18 and a series of rigid ratchet teeth 21 on the outer surface of inner skirt 19.

Inner screw thread 20 on each lid is the same as thread 8 on tray 1 and serves to co-operate with outer screw thread 11 on the neck or mouth of each container 2 so as to ensure a hermetic closure of the latter.

The radial distance between the outer skirt 18 and the inner skirt 19 of each lid is suitable to permit mouth 10 of the respective container to be received between them at the time of closure.

Ratchet teeth 21 have one gently sloping side and one stop side and are directed so as to flex ratchet tongues 12 on the neck and thus pass over them on the smoothly sloping side when the lid is screwed (clockwise) onto the container (ratchet action), but positively prevent unscrewing of the lid (anti-clockwise) due to the abutment of the free ends 14 of tongues 12 with the stop sides of teeth 21.

The strength of tongues 12 when an attempt is made of unscrewing against the stop surfaces of ratchet teeth 21 is greater that that provided by the weakened region 15 at the base of mouth 10. Consequently, any attempt at tampering by forced unscrewing of the closure lid will result in rupture of the mouth and its total or partial separation from the body 9 of the container, effectively destroying the latter and leaving it clearly evident that there has been an unauthorised attempt at opening.

When the dope test kit described above is to be used, the two containers 2 are screwed to the bottom of tray 1. The urine sample collected in the tray automatically passes to the two containers that are then unscrewed and closed with the respective closure lids 3, using the co-operation both of screw threads 11 and 20 and of ratchet elements 12 and 21. The containers may be handled using the side grips or handles that are shown in the drawings. The containers are then transported to the analysis location where one of them is opened by breaking the protuberance 16 at the bottom of the container. The other container is saved for a second analysis should the results of the first one indicate some irregularity, thus ensuring a trustworthy result.

It will be understood immediately that the construction of containers 2 and their closures 3 provide a high degree of security against attempts of violation due to the fact that the ratchet elements—the tongues 12 and the ratchet teeth 21—are hidden within the container-lid combination and, furthermore, are sandwiched within the narrow annular space between the outer surface of the inner skirt 19 on the lid and the inner surface of the mouth 10 of the container. This effectively prevents any undetectable violation by disengaging the ratchet elements from each other. On the other hand, the weakened region 15 ensures that inordinately large force applied in an attempt to unscrew the closure lid will result in destruction of the container, leaving clear signs of violation.

It is to be understood that the above description relates to a presently preferred embodiment of this invention and that the specific structural details are not limitative to its scope which is defined in the accompanying claims. For example, it is essential—in accordance with the invention—that the ratchet elements be totally hidden within the closed container-lid assembly by way of there being an inner skirt on the lid which carries ratchet elements, combined with corresponding ratchet elements on the inner surface of the neck or mouth of the container.

Although the above description and the drawing show that a liquid sample may be removed from the container 2 by breaking a protuberance 16 at the underside of its base, it will be appreciated that other options could be used. For example, the sample or specimen in question could be faeces or other solid matter and could be contained in a small recipient placed within container 2. In such a case the breaking of the small protuberance 16 would not permit removal of the specimen. It would then be possible, for example, for the complete lower end of container 2 to be formed with a circumferential weakening so that twisting would result in total removal of the base of the container, allowing immediate removal of the contents. In his sense it will be appreciated that intentional opening with effective destruction of the base of the container, be it by complete removal thereof of by the breaking of the protuberance 16, will still leave the upper end intact so that tampering or the absence thereof can be checked at a later time should the result of any test be questioned. Naturally, it would be possible for a container 2 to have both the protuberance 16 and the circumferential weakening mentioned above so that it could be used either with just a liquid or with a specimen of larger physical proportions.

These and other minor modifications are considered to be within the scope of the present invention.

1. Container with a security closure comprising:
a hollow body (9) having a mouth (10) defining an opening to the interior of the body, the mouth having an outer thread (11) and also a first series of ratchet elements (12); and
a closure (3) adaptable to the mouth (10) of the container for closing the opening in the body, the closure having a closed end (17) and an outer skirt (18), the outer skirt being formed with an inner thread (20) co-operable with the outer thread (11) on the mouth (10), the closure also being formed with a second series of ratchet elements (21) so that, when the closure is screwed onto the mouth (10), the co-operation between the ratchet elements of the first and second series (12, 21) permits closure of the container but prevents unscrewing of the closure;
characterised in that:
the ratchet elements (12) of the first series are distributed circumferentially around the inner surface of the mouth (10);
the closure (3) is provided with an inner skirt (19) such that, when the container is closed, the mouth (10) fits between the outer and inner skirts (18, 19) of the closure; and
the ratchet elements (21) of the second series are distributed circumferentially around the outer surface of the inner skirt (19).

2. Container with a security closure according to claim 1, characterised in that the mouth (10) is joined to the body (9) of the container by means of a weakened circumferential region (15) that is less resistant to twisting forces between the mouth (10) and the body (9) than those necessary to unscrew the closure (3) from the mouth, against the resistance provided by the ratchet elements (12, 21).

3. Container with a security closure according to claim 1 or 2, characterised in that each ratchet element (12) of one of the series is flexible so as to allow passage of the ratchet elements (21) of the other series when the closure is screwed onto the mouth (10) of the container but, due to their abutment against the ratchet elements of the other series, resists bending when unscrewing is attempted.

4. Container with a security closure according to claim 3, characterised in that the ratchet elements of the second series comprise teeth (21) formed around the external surface of the inner skirt (19) and the ratchet elements of the first series are flexible tongues (12), each tongue being integral at one end (13) with the inner surface of the mouth (10) and having a free end that inter-engages with a sloping side of successive teeth (21) of the second series to flex the tongue (12) and thus to allow screwing on of the closure, and with a stop side of a respective tooth (21) to prevent bending of the tongue and thus not allow unscrewing of the closure.

5. Container with a security closure according to any one of claims 1 to 4, characterised in that the body (9) and the mouth (10), with its screw thread (11) and ratchet elements (12), comprise a single part of plastic and the closure (3) corn its skirts (18, 19), screw thread (20) and ratchet elements (21) comprise another single part of plastic.

6. Container with a security closure according to any one of claims 1 to 5, characterised in that the body (9) is formed, at a location remote from the mouth (10), with a weakened easily broken upstanding region (16) to provide an outlet opening for the contents of the body (9) without requiring removal of the closure (3).

7. Container with closure according to any claim 6, characterised in that the body (9) has a recessed part in its base, said weakened protuberant region (16) being located in the recessed part.

8. Kit for containing samples of urine, characterised by comprising
a urine receiving tray (1) having a base (4) formed with two interspaced orifices (6), each orifice (6) leading to a lower tubular portion (7) projecting downwardly from the lower surface of the base (4) and each tubular portion (7) having an internal thread (8);
two containers (2) for urine collected in the tray (1), each container (2) comprising a hollow body (9) having a mouth (10) defining an opening to the interior of the body, the mouth having an outer thread (11) co-operable with the thread (8) on either one of said lower tubular portions (7) of the tray, and also a first series of ratchet elements (12) distributed uniformly around the inner surface of the mouth (10); and
two closures (3), each adaptable to the mouth for closing the opening in either one of the two bodies (9), each closure comprising:
a closed end (17);
an outer skirt (18), the outer skirt being formed with an inner thread (20) co-operable with the outer thread (11) on the mouth (10) of either one of the bodies:
an inner skirt (19) positioned such that, when the container is closed, the mouth (10) fits between the outer and inner skirts (18, 19); and
a second series of ratchet elements (21) distributed circumferentially around the outer surface of said inner skirt (19), so that, when the closure (3) is screwed onto the mouth (10) of a respective body (9), the co-operation between the ratchet elements (12, 21) of the first and second series permits closure of the respective container but prevents unscrewing of the closure.

9. Kit for containing samples of urine, according to claim 8, characterised in that the mouth (10) of each container is connected to the body (9) by means of a weakened circumferential region (15) that is less resistant to twisting forces between the mouth (10) and the body (9) than those necessary to unscrew the closure from the mouth, against the resistance provided by the ratchet elements (12, 21).

10. Kit for containing samples of urine, according to claim 8 or 9, characterised in that each ratchet element (12) of one of said series, on each container, is flexible so as to allow the passage of the ratchet elements (21) of the other series upon screwing of the closure (3) onto the mouth (9) and, due to its abutment against the ratchet elements (21) of the other series, resists flexion upon a tentative to unscrew it.

11. Kit for containing samples of urine, according to claim 10, characterised in that the ratchet elements of the second series comprise teeth (21) formed around the external surface of the inner skirt (19) and the ratchet elements of the first series are flexible tongues (12), each tongue being integral at one end (13) with the inner surface of the mouth (10) and having a free end (14) that inter-engages with a sloping side of successive teeth (21) of the second series to flex the tongue (12) and thus to allow screwing on of the closure, and with a stop side of a respective tooth (21) to prevent bending of the tongue and thus not allow unscrewing of the closure.

12. Kit for containing samples of urine, according to any one of claims 8 to 11, characterised in that the body (9) and the mouth (10) of each container, with its thread (11) and ratchet elements (12), comprise a single part of plastic and each closure (3), with its skirts (18, 19), thread (20) and ratchet elements (21), comprise another single part of plastic.

13. Kit for containing samples of urine, according to any one of claims 8 to 12, characterised in that the body (9) of each container is formed, at a location remote form the mouth (10), with a weakened easily ruptured protuberant region (16) to provide an outlet opening for the contents of the body without there being any necessity to remove the closure (3).

14. Kit for containing samples of urine, according to claim 13, characterised in that the body (9) of each container has a recessed part in its base, said weakened protuberant region (16) being located in said recessed part.
